(19) United States
(12) Patent Application Publication
(10) Pub. No.: US 2026/0272268 A1
(43) Pub. Date: Sep. 17, 2026

(54) UNIVERSAL SCOPE ADAPTER FOR AN INTERVENTIONAL DEVICE

(71) Applicant: DRAGONFLY ENDOSCOPY INC., Englewood, CO (US)

(72) Inventors: Benjamin MORRIS, Jeffersonville, IN (US); Anthony APPLING, Crestwood, KY (US); Chase WOOLEY, Floyds Knobs, IN (US)

(21) Appl. No.: 19/669,824

(22) Filed: May 6, 2026

Related U.S. Application Data
(63) Continuation of application No. PCT/US2024/055231, filed on Nov. 8, 2024.
(60) Provisional application No. 63/547,749, filed on Nov. 8, 2023.

(51) Int. Cl. A61B 1/00; A61B 1/005; A61B 1/018

(52) U.S. Cl. CPC A61B 1/00128; A61B 1/00042; A61B 1/0052; A61B 1/018

(57) ABSTRACT

An apparatus for mating a diagnostic device to an interventional device having a control handle that extends along a main axis A. The control handle of the interventional device includes an instrument port extending outwardly along an off-axis A₁ disposed at an acute angle relative to the main axis A. A valve cap is mounted on the instrument port to present a defined outer geometry. The diagnostic device extends along a device axis A_{D} from a proximal device end to a distal device end, and a universal scope adapter is disposed at the distal device end. The universal scope adapter defines an internal mounting cavity sized complementary to and mated with the defined outer geometry of the valve cap to place the device axis A_{D} in axially aligned relationship with the off-axis A₁ and initially mount the diagnostic device in self-supporting relationship to the interventional device.

### CROSS-REFERENCE TO RELATED APPLICATION

This patent application claims priority to PCT/US2024/055231, filed Nov. 8, 2024 and published as WO2025/101975, which claims the benefit of U.S. Provisional Application Ser. No. 63/547,749 filed on Nov. 8, 2023, each of which is incorporated herein by reference in its entirety.

### TECHNICAL FIELD

The present disclosure relates to interventional devices such as those used in medical operations. More particularly, the present disclosure relates to an apparatus for use in mounting a therapeutic or diagnostic device to an interventional device such as an endoscope (e.g., duodenoscope, gastroscope, bronchoscope, therapeutic scope, colonoscope) or similar medical instrument.

### BACKGROUND OF THE DISCLOSURE

Interventional devices are used for visualizing surfaces inside objects. For example, an endoscope is a medical instrument for visualizing the interior of a patient's body. Endoscopes can be used for a variety of different diagnostic and interventional procedures, including colonoscopy, bronchoscopy, thoracoscopy, laparoscopy, ureteroscopy and video endoscopy. Endoscopes typically have a control handle which is configured to allow a user to control a position of a distal tip of an endoscope tube during the procedure to investigate for the presence of any undesirable objects, such as the presence of stones, polyps or tumors during an endoscopic procedure, as an example.

Endoscopes typically have an instrument port for allowing a variety of therapeutic or diagnostic devices (hereinafter in most cases referred to only as a “diagnostic device”) to be utilized with the endoscope and disposed in communication with a working channel of the endoscope tube. However, as noted above, there are a large variety of endoscopes (e.g., duodenoscope, gastroscope, bronchoscope, therapeutic scope, and colonoscope, as examples) each having unique and various physical features and dimensions at their point of entry to the endoscope. Each of these entry instrument ports also require different types of seals over the instrument port in use that is unique to the endoscope. Additionally, the present arrangements for mounting the diagnostic device to the endoscope are cumbersome, and often require more than one user to complete the process, with a first user holding both the diagnostic device and the interventional device while another second user secures the diagnostic device and interventional device to one another. Accordingly, there remains a need for an apparatus which can universally attach to the unique instrument ports for the majority, if not all, types of interventional devices and which can provide a simpler way to mount and secure a variety of different therapeutic or diagnostic devices to the interventional device (e.g., endoscope) using only a single user.

### SUMMARY OF THE INVENTION

An apparatus for mating a diagnostic device to an interventional device having a control handle that extends along a main axis A. The control handle of the interventional device includes an instrument port extending outwardly along an off-axis A₁ disposed at an acute angle relative to the main axis A. A valve cap is mounted on the instrument port to present a defined outer geometry. The diagnostic device extends along a device axis A_{D} from a proximal device end to a distal device end, and a universal scope adapter is disposed at the distal device end. The universal scope adapter defines an internal mounting cavity sized complementary to and mated with the defined outer geometry of the valve cap to place the device axis A_{D} in axially aligned relationship with the off-axis A₁ and initially mount the diagnostic device in self-supporting relationship to the interventional device.

The universal scope adapter advantageously allows a user to remove their hands from the diagnostic device to leave the diagnostic device hanging on the interventional device under its own weight and gravity (i.e., self-supporting) without the need of the user to support the diagnostic device (i.e., hands-free). Put another way, the universal scope adapter on the diagnostic device advantageously allows the user to manually support only the interventional device after the initial mounting of the diagnostic device, since the diagnostic device will hang relatively or absolutely static in relation to the interventional device oriented in normal use.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1 is a perspective view of an exemplary endoscope as the interventional device;

FIG. 2 is an exploded perspective view illustrating a universal scope adapter disposed on a distal device end of a diagnostic device that extends along a device axis A_{D} and is disposed in axially aligned relationship with an off-axis A₁ defined by an instrument port of a control handle of the endoscope;

FIG. 3 is a fragmentary cross-sectional view of the diagnostic device and the endoscope in separated and non-mounted relationship with one another to illustrate an internal mounting cavity defined by the universal scope adapter and sized complementary to a common outer geometry of the valve cap;

FIG. 4 is a magnified fragmentary cross-sectional view of the diagnostic device and the endoscope disposed in an initial mounted relationship with one another to illustrate a custom inner geometry of the valve cap sized complementary to and mated with the instrument portion and the internal mounting cavity disposed in surrounding and sealed relationship with the common outer geometry of the valve cap;

FIG. 5 is the fragmentary cross-sectional view of FIG. 3 with the valve cap removed from the endoscope to more clearly illustrate the instrument port;

FIG. 6 is the fragmentary cross-sectional view of FIG. 4 with the valve cap removed from the endoscope to more clearly illustrate the instrument port relative to the internal mounting cavity when the universal scope adapter is mounted to the endoscope;

FIG. 7 is a fragmentary perspective view of the diagnostic device initially mounted to and disposed on the endoscope in self-supporting relationship (i.e., under its own weight), and illustrating a securement band, a translating component and a housing component of the universal scope adapter for use in subsequently securing the diagnostic device to the endoscope;

FIG. 8 is a fragmentary perspective view of the diagnostic device and the endoscope illustrating a sequential step of wrapping the the securement band around the endoscope and into releasably connected relationship with the translating component to establish a closed loop of the securement band extending around the control handle of the endoscope to secure the diagnostic device to the endoscope;

FIG. 9 is a fragmentary perspective view of the diagnostic device and the endoscope illustrating a subsequent step of translating (i.e., linearly advancing) the translating component along a securement plane relative to the translating component and the universal scope adapter from an initial position to an advanced position to decrease an inner diameter of the closed loop and tighten the securement band around the control handle of the endoscope for completing attachment of the diagnostic device to the endoscope;

FIG. 10 is a fragmentary perspective view of the diagnostic device and the endoscope illustrating a final step of compressing a pair of disengagement features to back a pair of fixation features away from respective locking shoulders to allow the translating component to be released from the advanced position and translated back to the initial position to loosen and remove the securement band wrapped around the control handle of the endoscope;

FIG. 11A illustrates an exemplary and non-limiting valve cap including a bifurcated lid defining a slit to provide a pneumatic seal about the instrument port;

FIG. 11B illustrates a side cross-sectional view of the exemplary valve cap of FIG. 11A;

FIG. 11C illustrates a top view of the valve cap of FIG. 11A; and

FIG. 11D illustrates a side view of the exemplary valve cap of FIG. 11A.

### DETAILED DESCRIPTION OF THE EXEMPLARY EMBODIMENTS

Example embodiments will now be described more fully with reference to the accompanying drawings. In general, the subject embodiments are directed to an apparatus for mating a diagnostic device to an interventional device. However, the example embodiments are only provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, and that example embodiments may be embodied in many different forms and that neither should be construed to limit the scope of the disclosure. In some example embodiments, well-known processes, well-known device structures, and well-known technologies are not described in detail.

Referring to the Figures, wherein like numerals indicate corresponding parts throughout the several views, an interventional device 10 is generally shown. According to the example embodiment, the interventional device 10 is shown and described as an endoscope, and thus these terms may be used interchangeably. However, the exemplary description of the endoscope 10 is not limiting, and the teachings herein may be applied to other types of interventional devices 10 without departing from the scope of the disclosure and invention. Moreover, the endoscope 10 shown in the Figures is designed to be utilized in association with various diagnostic and interventional procedures, such as via use of the universal scope adapter 30 which will be described in more detail below.

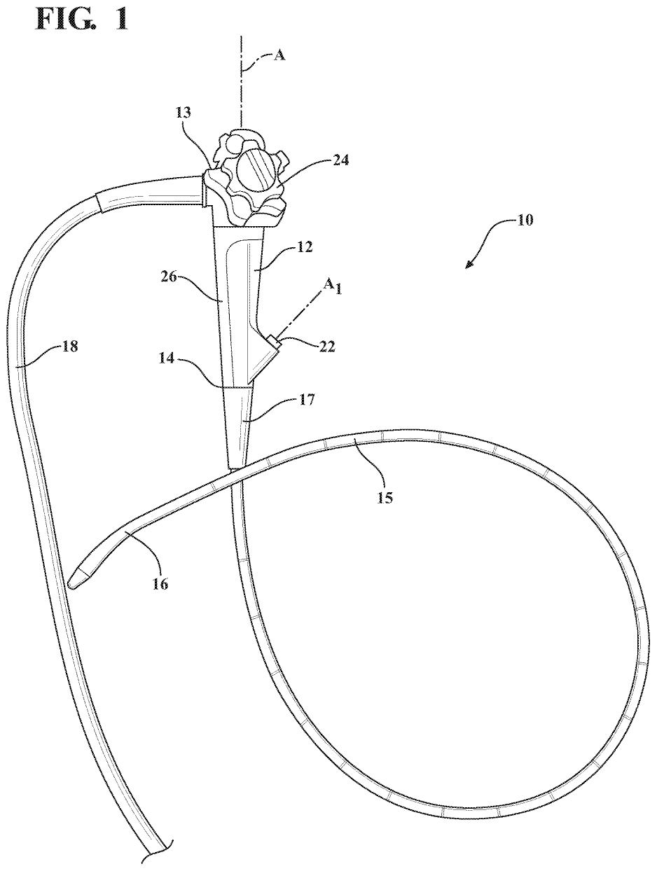

As illustrated in FIG. 1, the endoscope 10 includes a control handle 12 extending along a main axis A from a proximal handle end 13 to a distal handle end 14. An endoscope tube 15 extends from the control handle 12, adjacent the distal handle end 14, and terminates at a distal tip 16 for being located inside a patient's body for diagnostic and interventional procedures. A strain relief 17 can surround the endoscope tube 15 at an interface of the endoscope tube 15 and the control handle 12 to provide flexibility to, and for protecting, the endoscope tube 15. An umbilical cable 18 extends from the control handle 12 for being coupled with a processing device for evaluating data obtained by the distal tip 16. An instrument port 22 is located on and extends outwardly from the control handle 12 along an off-axis A₁ to provide access to the working channel of the endoscope tube 15 that extends along the main axis A and is located between the instrument port 22 and the distal tip 16. As best illustrated in FIGS. 1-6, the off-axis A₁ is disposed at an acute angle relative to the main axis A. As will be described in more detail below, the instrument port 22 allows attachment of user prescribed diagnostic devices 23 and provides access for insertion of tools and irrigation. With further reference to FIG. 1, a steering assembly 24 can be provided on the control handle 12 for allowing a user to control movement of the distal tip 16 of the endoscope tube 15 during the diagnostic and interventional procedure. A gripping region 26 can be located between the proximal and distal ends 13, 14 of the control handle 12 and is shaped to receive a palm and fingers of a user to provide easy, comfortable gripping of the control handle 12 by the user during use of the steering assembly 24.

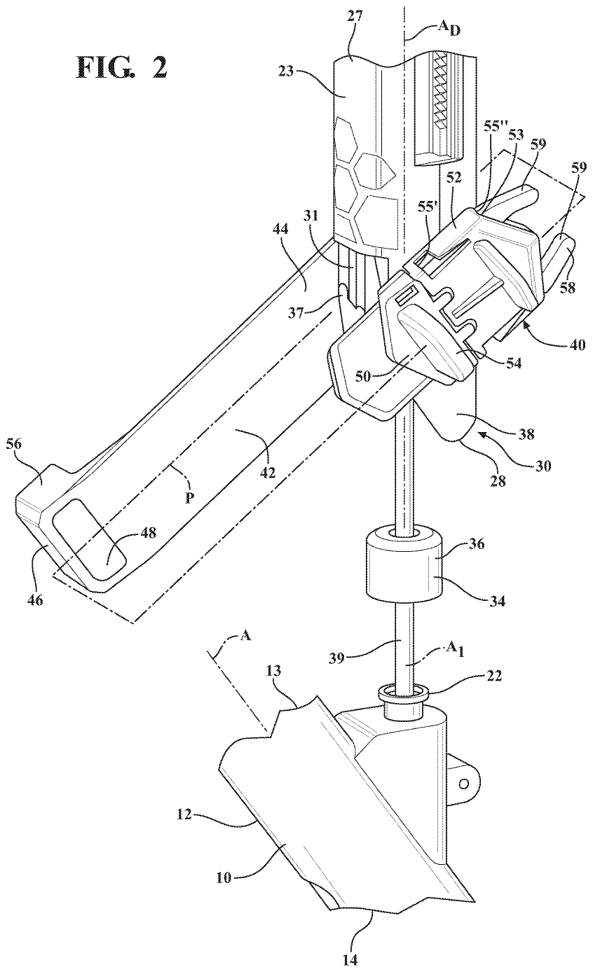

As mentioned previously, the therapeutic or diagnostic device 23 is ultimately attached to the endoscope 10 and disposed in communication with the working channel of the control handle 12 and the endoscope tube 15 to perform a specific therapeutic or diagnostic procedure. For example, the therapeutic of diagnostic device can be tools such as fine needle aspiration devices, steerable biopsy devices, endoscopic tools that require linear and radial manipulation with stability, or the like. As illustrated in FIGS. 2-10, the diagnostic device extends along a device axis A_{D} from a proximal device end 27 to a distal device end 28. A universal scope adapter 30 is disposed at the distal device end 28 of the diagnostic device 23 and is configured to be mounted on the instrument port 22 for allowing a variety of therapeutic or diagnostic devices 23 to be universally coupled with a variety of endoscopes, depending on the desired therapeutic or diagnostic procedure. In other words, as will be described in more detail immediately below, the universal scope adapter 30 provides for a mounting and securement device which advantageously allows a variety of therapeutic or diagnostic devices to be easily mounted to and coupled with a plurality of different endoscopes in a simpler manner by a single person.

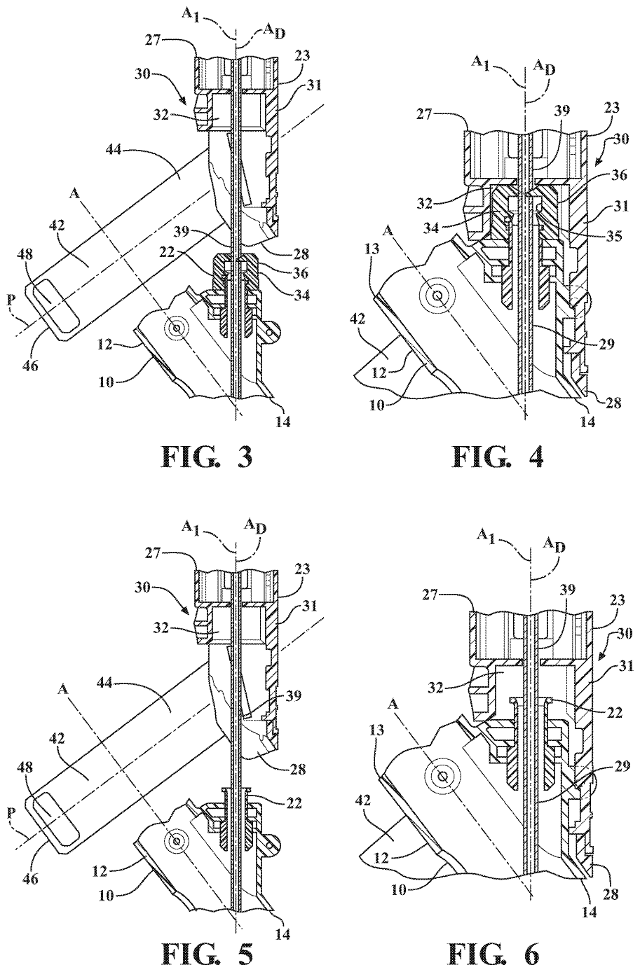

While an instrument port 22 is common and of similar form among endoscopes, a fitting that accepts a valve in the instrument port 22 is often of different forms across various endoscope models. Accordingly, in a preferred arrangement of the universal scope adapter 30, and as best illustrated in FIG. 2-4, a valve cap 34 is mounted on the instrument port 22 and defines a custom inner geometry 35 (See FIG. 4) sized complementary to the instrument port 22 to properly mate and seal the valve cap 34 with the specific and unique endoscope model. As further illustrated in FIGS. 2-4, the valve cap 34 also presents a common outer geometry 36 of a pre-defined size and shape (in this case cylindrical) which is shared among all of the variations of the valve cap 34. Put another way, while the valve cap 34 can include varying cross-sectional shapes for the custom inner geometry 35 depending on the instrument port 22 to which the valve cap 34 will be mated, the common outer geometry 36 of the valve cap 34 is shared across all of the various valve caps 34. As best illustrated in FIGS. 3-6, a main body 31 of the universal scope adapter 30 defines an internal mounting cavity or pocket 32 that is sized complementary to the common outer geometry 36 of the valve cap 34. Although described and illustrated as being sized complementary to the valve cap 34, the use of the valve cap 34 is non-limiting and if not present, the internal mounting cavity or pocket 32 of the universal scope adapter 30 could be sized complementary to an outer geometry of the instrument port 22 without departing from the scope of the disclosure.

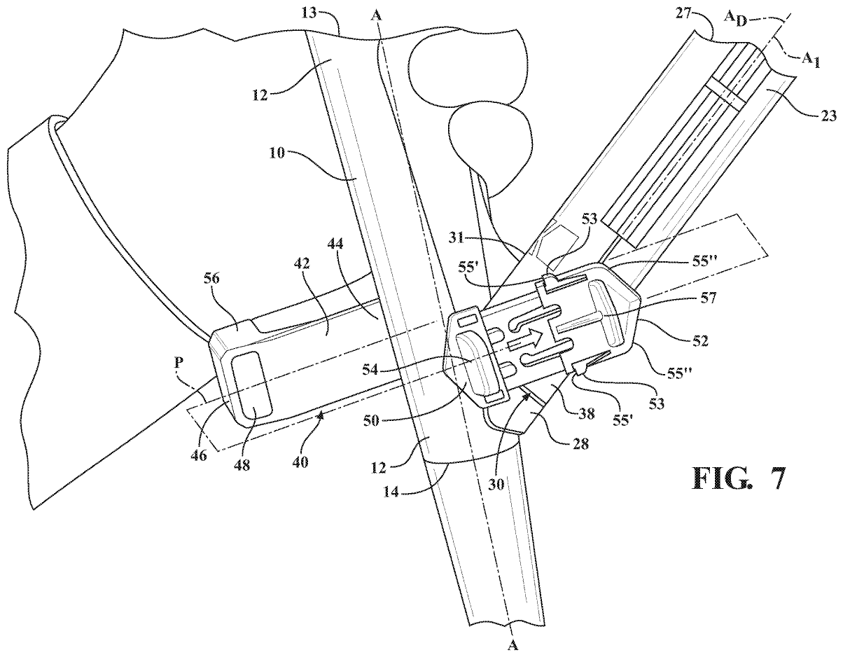

In either arrangement, the complementary size of the internal mounting cavity 32 relative to the common outer geometry 36 of the valve cap 34 (or the instrument port 22, if the valve cap 34 is not utilized) provides a physical feature for allowing an initial stage/interface of mounting the diagnostic device 23 to the interventional device 10 to be “hands free” and self-supporting. More specifically, as illustrated in FIGS. 2-4, the diagnostic device 23 with the universal scope adapter 30 on the distal device end 28 is initially advanced along the off-axis A₁ and placed over the instrument port 22 such that the internal mounting cavity 32 seats over the valve cap 34 disposed on the instrument port 22 to initially mount the diagnostic device 23 to the endoscope 10 in self-supporting and axially aligned relationship with and along the off-axis A₁ (i.e., with the device axis A_{D} axially aligned with the off-axis A₁, as shown in FIGS. 2-4). In this initial mounting of the diagnostic device 23 to the endoscope 10, the internal mounting cavity 32 of the universal scope adapter 30 is disposed in surrounding and sealed relationship with the valve cap 34 on the instrument port 22 such that the diagnostic device 23 hangs on and is self-supported relative to the endoscope 10, oriented as in normal use. In other words, once the diagnostic device 23 is initially mounted on the instrument port 22 of the endoscope 10, the physical interaction between the internal mounting cavity 32 and the valve cap 34 as well as the axially aligned relationship between the device axis A_{D} and the off-axis A₁ allows a user to remove their hands to leave the diagnostic device 23 hanging on the endoscope 10 under its own weight and gravity (i.e., self-supporting) without the need of the user to support the diagnostic device 23 (i.e., hands-free). This advantageously allows the user to manually support only the endoscope 10 after the initial mounting of the diagnostic device 23 (See FIG. 7), since the diagnostic device 23 will hang relatively or absolutely static in relation to the endoscope 10 oriented in normal use.

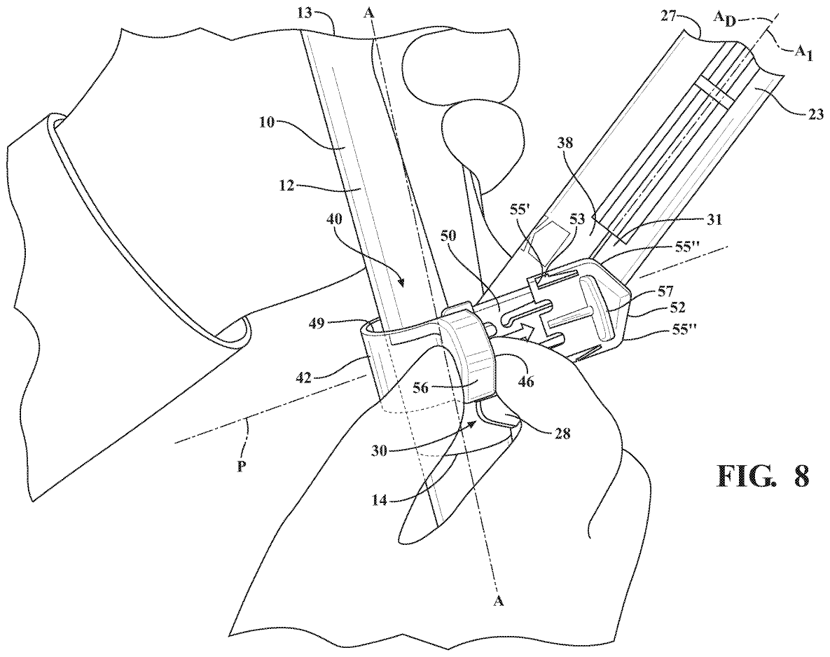

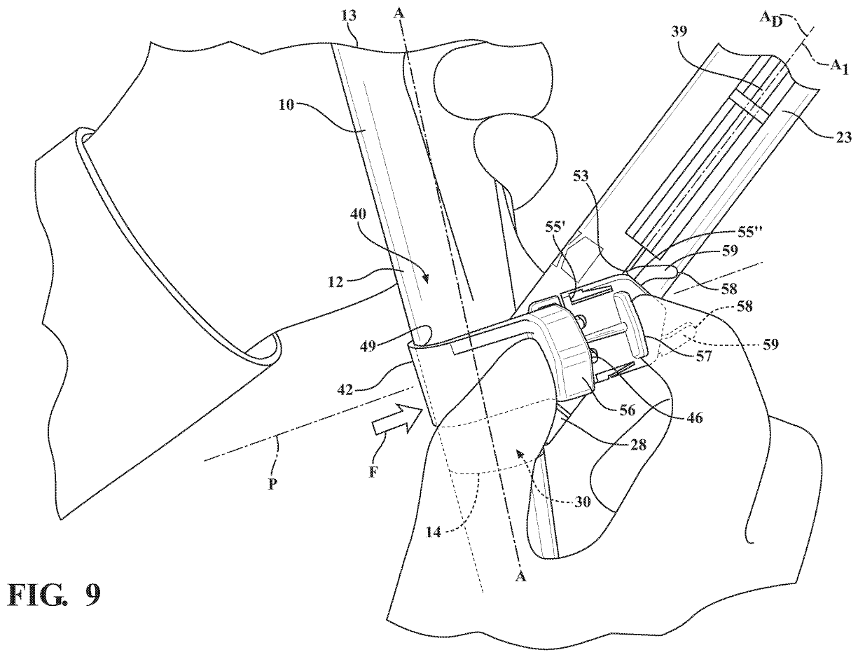

As will be appreciated in view of the following disclosure, this allows the user to perform additional steps of coupling the diagnostic device 23 to the endoscope 10 with one hand (See FIGS. 8-9) and without the need to support both the diagnostic device 23 and the endoscope 10 each individually (which can be cumbersome and difficult based on the size and orientation of the diagnostic device 23 and endoscope 10) or with assistance from other users. Put another way, the universal scope adapter 30 allows the diagnostic device 23 to be initially mounted and secured directly to the endoscope 10 so that a single user can operate both the endoscope 10 and the diagnostic device 23 as an assembly during coupling with just two hands. (See FIGS. 7-10). And as further illustrated in FIGS. 3-6, the axially aligned relationship of the device axis A_{D} and the off-axis A₁ positions the diagnostic device 23 on-axis and aligned with the instrument port 22 such that a device catheter 29 and/or guide wire 39 passing through the diagnostic device 23 during use can directly access the instrument port 23 and the ensuing working channel of the control handle 12 and endoscope tube 15, i.e., without the need for a service loop passing externally to the diagnostic device 23 to establish this access of the device catheter and/or guide wire to the instrument port and the working channel.

Since this initial mounting interface may actualize as a loose attachment due to the variety of physical geometries of the endoscope 10, as best illustrated in FIGS. 2 and 7-9, the universal scope adapter 30 includes a securement assembly 40 for establishing a secure fit of the diagnostic device 23 to the endoscope 10 after the initial mounting step shown in FIGS. 4 and 7. As best illustrated in FIG. 2, the main body 31 of the universal scope adapter 30 includes a first side 37 and a second side 38 disposed opposite the first side 37. Furthermore, in a preferred arrangement, the securement assembly 40 includes a securement band 42 comprised of an elastomeric material that when in use extends from a first band end 44 fixed to the first side 37 of the universal scope adapter 30, is wrapped around the control handle 12 of the endoscope 10, and terminates at a second band end 46 that is releasably secured to the second side 38 of the universal scope adapter 30 to establish a closed loop 49 around the control handle 12 for tightening or enhancing the initial mount of the diagnostic device 23 to the interventional device 10. Put another way, the securement band 42 is not required to maintain the diagnostic device 23 on the endoscope 10, but rather functions to wrap a closed loop 49 of the securement band 42 around the control handle 12 to absorb differences in geometry of various control handles/endoscopes, and enhance attachment of the diagnostic device 23 to the interventional device 10.

As best illustrated in FIGS. 7-9, the securement band 42 is fixed to the first side 37 of the universal scope adapter 30 such that it is arranged and extends along a securement plane P_{S} that is disposed transversely relative to the main axis A and at an obtuse or acute angle (depending on point of reference) relative to both the off-axis A₁ and device axis A_{D} to ultimately apply a compressive force F transversely to the control handle 12 in relation to its main axis A. (See FIG. 9). Applicant found that this transverse compressive force F advantageously provides a more stable mounting of the diagnostic device 23 relative to the interventional device 10, which “pulls” the diagnostic device 23 down against the interventional device 10 at the instrument port 10 and the working channel. Put another way, Applicant found that application of the transverse compressive force F is critical to the mounting stability of the diagnostic device 23 on the endoscope 10. In contradistinction, Applicant found that application of a non-transverse compressive force by the securement band 42 initially squeezes the control handle 12 but ultimately results in the diagnostic device 23 raising its position relative to the endoscope 10 during use, resulting in a less stable and non-ideal mount with less compression applied by the securement band 42.

As best illustrated in FIG. 7, the securement band 42 extends to the second band end 46 which before being wrapped around the control handle 12 is initially free and presents a socket feature 48. As further illustrated in FIGS. 7-9, the securement assembly 40 includes a translating component 50 arranged adjacent and indirectly connected to the second side 38 of the main body 31 of the universal scope adapter 30. More specifically, as will be explained in more detail immediately below, the translating component 50 is at least partially housed within and translatable relative to a housing component 52 fixed to the second side 38 of the universal scope adapter 30. As best illustrated in FIGS. 2 and 7, the translating component 50 defines a plug feature 54 that is sized complementary to the socket feature 48 of the securement band 42. For example, the plug feature 54 can be designed as a male component with the socket feature 48 correspondingly defined as a female component. However, as will be appreciated in view of the following description, the arrangement of the male and female components could be reversed, without departing from the scope of the subject disclosure.

As best illustrated in FIGS. 8-9, and as discussed previously, the securement band 42 is designed with flexibility and dimensions such that it can be wrapped by the user from the first side 37 of the universal scope adapter 30, around an exposed portion of the control handle 12 of the endoscope 10, and to the opposite second side 38 of the universal scope adapter 30 to dispose the socket feature 48 of the securement band 42 in releasably mated relationship with the plug feature 54 of the translating component 50. Put another way, by attaching the socket feature 48 of the securement band 42 to the complementary plug feature 54 of the translating component 50, the open loop of the securement band 42 is wrapped around the endoscope 10 to establish the closed loop 49 (See FIGS. 8-9), and the releasably secured relationship of the second band end 46 to the second side 38 of the universal scope adapter 30. And notably, there is little to no tension/resistance in installing the securement band 42 to the translation component 50 and completing the closed loop 49 around the endoscope 10 in the initial position. From a user and ergonomic perspective, this step requires no gross hand strength or dexterity, enabling the user population to be as large as possible for comfortable operation of the universal scope adapter 30.

However, in this initial coupling of the socket feature 48 with the plug feature 54, the securement band 42 may only be loosely closed and looped around the control handle 12 of the endoscope 10. Accordingly, as best illustrated in sequential FIGS. 8-9, the translating component 50 is translatable (i.e., linearly advanced) along the securement plane P and relative to the housing component 52 and the main body 31 of the universal scope adapter 30 to which the housing component 52 is fixed from an initial position (shown in FIG. 8) to at least one advanced position (as shown in FIG. 9) to decrease an inner diameter of the closed loop 49 around the endoscope 10 and effectively tighten a securement of the diagnostic device 23 to the endoscope 10. Put another way, in a preferred embodiment, the translating component 50 can be cycled or translated linearly along the securement plane P between a plurality of fixed positions relative to the housing component 52 and the second side 38 of the universal scope adapter 30 until an appropriate tightness of the securement band 42 relative to the endoscope 10 is established. By cycling the translating component 50 from an initial start position (FIG. 8) to at least one advanced position (FIG. 9), the user is able to decrease the inner diameter of the closed loop 49 of the securement band 42 incrementally for constricting the securement band 42 around the endoscope 10 to minimize any remaining relative looseness between the diagnostic device 23 and the endoscope 10. This capability allows the translating component 50 to be used with a variety of endoscopes, since the fixed housing component 52 is located at different distances from the mated endoscope 10 depending on its unique geometry. And notably, since the diagnostic device 23 is initially self-supported relative to the endoscope 10 by way of the internal mounting cavity 32, this cycling of the translating component 50 can be accomplished by a single user, namely because support of the diagnostic device 23 separately from the endoscope 10 is not required. (See FIGS. 8-9).

As best illustrated in FIGS. 8-9, the translating component 50 can be held in these various positions by at least one fixation feature 53 (in this case preferably implemented as a pair of outwardly extending locking tabs 53 disposed in aligned relationship with one another on opposite sides of the translating component 50 and the securement plane P) that sequentially mates with a plurality of locking shoulders 55 (arranged sequentially and in spaced relationship with one another along the housing component 52) as the translating component 50 is first disposed in the initial position and then translated or linearly advanced to the at least one advanced position. More specifically, as best shown in FIG. 8, the housing component 52 defines at least one initial shoulder 55′ for mating with the at least one fixation feature 53 when the translating component 50 is disposed in the initial position, and the socket feature 48 of the securement band 42 is initially mated with the plug feature 54 of the translating component 50. The mating relationship of the at least one fixation feature 53 with the at least one initial shoulder 55′ maintains the closed loop of the securement band 42 in the initial position. As best shown in FIG. 9, the housing component then defines at least one advanced locking shoulder 55″ for subsequently mating with the at least one fixation feature 53 when the translating component 50 is sequentially translated and linearly advanced to the at least one advanced position. The mating relationship of the at least one fixation feature 53 with the at least one advanced shoulder 55″ maintains the tightened closed loop of the securement band 42 around the control handle 12. In a preferred arrangement, the plurality of locking shoulders 55 includes a pair of initial locking shoulders 55′ disposed in aligned relationship with one another on opposite sides of the housing component 52 and the securement plane P as well as a pair of advanced locking shoulders 55″ again disposed in aligned relationship with one another on opposite sides of the housing component 52 and the securement plane P. Thus, in this preferred embodiment, the pair of outwardly extending locking tabs 53 are disposed in locked or abutting relationship with a respective one of the pair of initial locking shoulders 55′ when the translating component 50 is disposed in the initial position, and then translated into locked or abutting relationship with a respective one of the pair of advanced locking shoulders 55″ when the translating component is linearly advanced to the at least one advanced position.

Additionally, as best illustrated in FIGS. 7-9, the securement band 42 includes a band projection 56 projecting outwardly from the second band end 46 in opposing relationship to the socket feature 46 for allowing the user to easily translate the translating component 50 (which is mated with the securement band 42) along the securement plane P from the initial position (FIG. 8) to the at least one advanced position (FIG. 9), such as by pushing the band projection 56 with a thumb or other finger on the hand that is not holding the endoscope 10. (See FIG. 8). Further, the housing component 52 includes a housing projection 58 projecting outwardly from the housing component 52 and away from the second side 38 of the universal scope adapter 30. The housing projection 57 in combination with the band projection 56 enables a biomechanical advantage by allowing a user to utilize a pinch grip (i.e., squeezing the band projection 56 and the housing projection 57 together with a thumb and index finger, as an example) to translate the translating component 50 and increase the tension of the securement band 42 around the endoscope. (See FIG. 9).

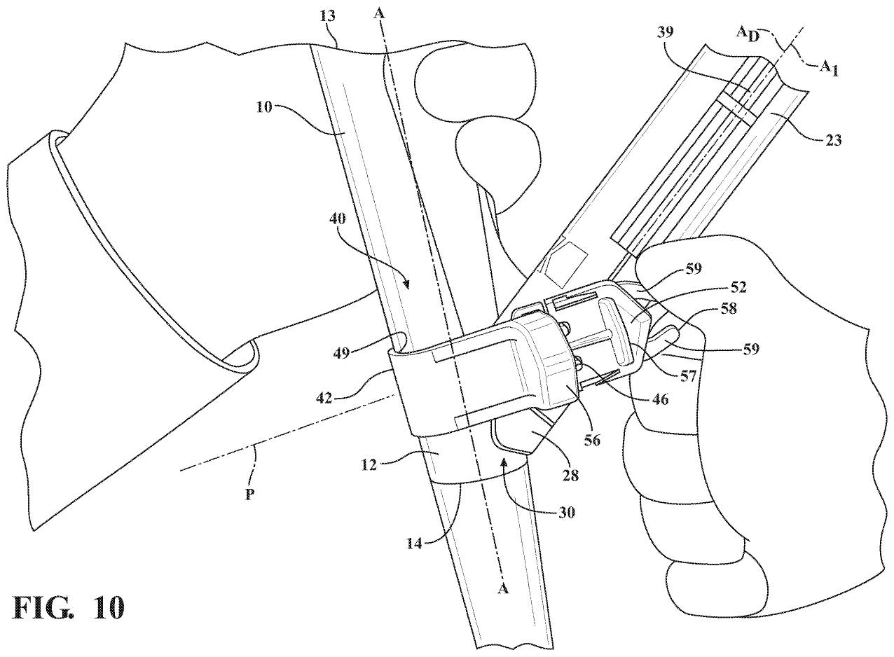

As best illustrated in FIG. 10, the securement assembly 40 includes a disengagement feature 58 that when engaged or actuated releases the translating component 50 from the at least one advanced position (shown in FIG. 9) and allows the user to return the translating component 50 to the initial position (shown in FIG. 8) in which little or no elastic tension is present in the securement band 42. For example, in a preferred embodiment, the disengagement feature 58 includes a pair of disengagement arms 59 which are disposed on the translating component 50 and extend outwardly from the housing component 52 when the translating component 50 is disposed in the at least one advanced position. The disengagement arms 59 are squeezed and advanced towards one another by the user to allow the pair of locking tabs 53 to be backed out of and free from the corresponding pair of advanced shoulders 55″, allowing the translating component 50 to translate back relative to the housing component 52 towards and into the initial position. The securement band 42 can then be removed from the translating component 50 and unwrapped from around the endoscope 10, after which the diagnostic device 23 can be removed from the instrument port 22 of the endoscope 10. However, it is also possible for the user to manually remove the socket feature 48 of the securement band 42 from the plug feature 54 of the translating component 50 while the securement band 42 is under tension in the advanced, fixed position, if they so choose, to open the closed loop 49 without cycling the translating component 50 back to the initial, starting position.

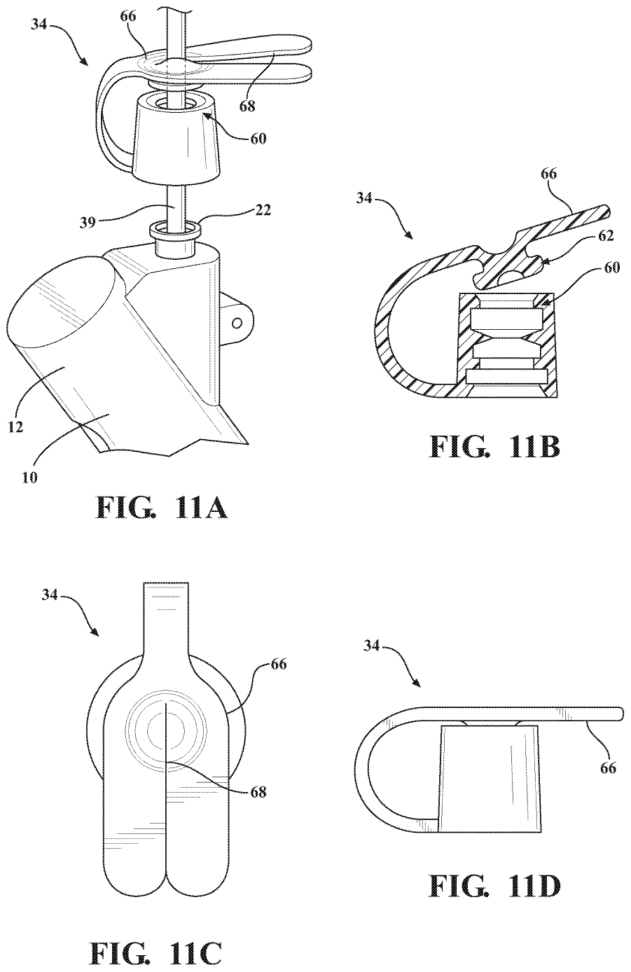

As best illustrated in FIGS. 11A-D, an exemplary arrangement of the valve cap 34 can feature a bifurcated lid 66 defining a slit 68 that provides a pneumatic seal about the instrument port 22. The slit 68 allows passage of smaller diameter tools, such as the guide wire 39 shown in FIGS. 2 and 11A, through the valve cap 34 while maintaining pneumostasis. The seal achieved by the slit 68 is maintained prior to being flipped back, as described below, by an uninterrupted diameter 60 of the body of the valve cap 34. The diameter of the valve cap 34 relative to a lid seal diameter 62 results in an interference condition when seated. This interference is complimented by the uninterrupted diameter 60 of the body providing hoop strength to the lid seal, and therefore squeezing the valve cap 34 along the bifurcation to maintain a seal. The area at the root of the slit 68 allows for passage of smaller tools, as is a common feature with these types of devices. As illustrated in FIGS. 11A-B, to facilitate passage of larger diameter tools, the bifurcated lid 66 can be flipped back to expose a secondary larger diameter inner valve over the instrument port 22. It is understood that due to the custom nature of each valve, the diameter of the bifurcated cap 66 and that of the inner valve can be a plurality of sizes to optimize passage of specific tools for specific procedures. Due to the bifurcation in the cap 66 as defined by the slit 68 this step can be performed without the need to remove any guide wire 39 that is present, enabling larger therapeutic or diagnostic devices to pass over the guide wire 39 while maintaining distal position and control of the guide wire 39 and minimizing the loss of the pneumostasis. The bifurcated lid 66 can also be flipped back and secured into the diameter of the valve cap 34, resealing the device or resealing around a tool such as a guide wire 39. Thus, as will be appreciated in view of the above-disclosure, in a preferred embodiment, the first step in utilizing the universal scope adapter 30 is selecting and installing the proper custom valve 34 to the endoscope 10.

In operation, and in more detail relative to a method of coupling a diagnostic device 23 to an interventional device 10, after the valve cap 34 having the common outer geometry 36 is installed on the instrument port 22, the therapeutic or diagnostic device 23 with the universal scope adapter 30 arranged at the distal device end 28 can be fed over a guide wire 39 (such as shown in FIG. 2) or directly onto the instrument port 22. In either arrangement, the therapeutic or diagnostic device 23 with the universal scope adapter 30 is advanced to the instrument port 22 so that the internal mounting cavity 32 defined by the universal scope adapter 30 is seated on the instrument port 22 and over the valve cap 34, such as shown in FIG. 4. At this point, the universal scope adapter 30 allows the device 23 to “hang” on the endoscope 10 in a “hands free” and self-supporting arrangement, with the device axis A_{D} disposed in axially aligned relationship with the off-axis A₁ defined by the instrument port 22. The securement band 42 of the securement assembly 40 is then wrapped around the control handle 12 of the endoscope 10 and the socket feature 42 adjacent to the second band end 46 is hooked or mated to the plug feature 54 on the translating component 50, after which the translating component 50 is translated relative to the housing component 52 and the universal scope adapter 30 along the securement plane P from an initial position to at least one advanced position to constrict the securement band 42 around the endoscope 10. Removal of the securement band 42 can later be achieved by actuating the disengagement feature 58 to release the translating component 50 from the at least one advanced position and allow the translating component 50 to translate relative to the housing component 52 back to the initial position for releasing the attendant tension applied to the control handle 12 by the securement band 42. This then allows the user to unwrap the securement band 42 from around the endoscope 10, after which the diagnostic device 23 can be removed away from the endoscope 10.

Obviously, many modifications and variations of the present disclosure are possible in light of the above teachings and may be practiced otherwise than as specifically described.

What is claimed is:

1. An apparatus for mating an interventional device to a diagnostic device extending from a proximal device end to a distal device end along a device axis, the system comprising:
a universal scope adapter disposed at the distal device end of the diagnostic device and configured to mount the diagnostic device in self-supporting relationship to the interventional device,
wherein the interventional device is an endoscope comprising:
a control handle extending from a proximal handle end to a distal handle end along a main axis;
an endoscope tube extending from the control handle adjacent the distal handle end and terminating at a distal tip; and
a steering assembly configured to control movement of the distal tip of the endoscope tube.

2. The apparatus of claim 1, wherein the endoscope further comprises an instrument port located on and extending outwardly from the control handle along an off-axis to provide access to a working channel of the endoscope tube.

3. The apparatus of claim 2, wherein the working channel of the endoscope tube extends along the main axis and is located between the instrument port and the distal tip.

4. The apparatus of claim 2, wherein the universal scope adapter is configured to place the device axis in axially aligned relationship with the off-axis.

5. The apparatus of claim 4, wherein the off-axis is disposed at an acute angle relative to the main axis.

6. The system of claim 2, further comprising a valve cap mounted on the instrument port and defining an outer geometry,
wherein the universal scope adapter defines an internal mounting cavity sized complementary to and mated with the defined outer geometry of the valve cap.

7. The system of claim 6, wherein the valve cap includes a bifurcated lid defining a slit to provide a pneumatic seal about the instrument port.

8. The system of claim 6, wherein the valve cap defines a custom inner geometry sized complementary to and mated with the instrument port to establish a sealed relationship therebetween.

9. The apparatus of claim 1, wherein the universal scope adapter further comprises a securement band extending from a first band end fixed to a first side of the universal scope adapter and wrapping around said control handle to a second band end releasably secured to a second side of the universal scope mount to establish a closed loop extending around the control handle to mount the diagnostic device in self-supporting relationship to the interventional device.

10. The apparatus of claim 9, wherein the securement band is configured to apply a compressive force on the control handle transversely to the main axis.

11. The apparatus of claim 10, wherein the securement band is arranged along a securement plane disposed at a transverse angle relative to the main axis and an obtuse angle relative to both the off-axis and the device axis to ultimately apply the compressive force on the control handle applied transversely to the main axis.

12. The apparatus of claim 9, wherein the universal scope adapter further comprises:
a housing component fixed to the second side of the universal scope adapter; and
a translating component at least partially housed within and translatable relative to the housing component from an initial position to at least one advanced position to decrease an inner diameter of the closed loop extending around the control handle.

13. The apparatus of claim 12, wherein the translating component includes a plug feature and the second band end of the securement band includes a socket feature configured to be disposed in mated relationship with the plug feature to establish the releasably secured relationship of the second band end with the second side of the universal scope adapter.

14. The apparatus of claim 12, wherein the housing component defines a plurality of locking shoulders arranged sequentially and in spaced relationship with one another relative to a securement plane, and wherein the translating component includes at least one fixation feature configured to sequentially mate with a respective one of said plurality of shoulders when the translating component is initially disposed in the initial position and then translated to the at least one advanced position to maintain the translating component in each of the initial position and the at least one advanced position.

15. The apparatus of claim 14, wherein the universal scope adapter further comprises a disengagement feature configured to release the at least one fixation feature from the respective shoulder when the translating component is disposed in the at least one advanced position for allowing the translating component to be returned to the initial position.

16. The apparatus of claim 15, wherein the at least one fixation feature is comprised of a pair of outwardly extending locking tabs disposed on the translating component in aligned and opposing relationship to one another, and wherein the plurality of locking shoulders include a pair of initial locking shoulders disposed in aligned and opposing relationship to one another and a pair of advanced locking shoulders disposed in aligned and opposing relationship to one another.

17. The apparatus of claim 16, wherein the disengagement feature includes a pair of disengagement arms disposed on the translating component and extending outwardly from the housing component when the translating component is disposed in the at least one advanced position for allowing the disengagement arms to be squeeze toward one another and back the pair of locking tabs out of and free from the respective pair of advanced locking shoulders.

18. The apparatus of claim 13, wherein the securement band includes a band projection projecting outwardly from the second band end in opposing relationship to the socket feature for allowing the band projection to be pushed and translate the translating component from the initial position to the at least one advanced position.

19. The apparatus of claim 18, wherein the housing component includes a housing projection projecting outwardly from the housing component and away from the second side of the universal scope adapter for allowing the band projection to be pinch gripped and the housing projection to translate the translating component relative to the housing component from the initial position to the at least one advanced position.

20. The apparatus of claim 9, wherein the securement band is comprised of an elastomeric material.
